(19) United States
(12) Patent Application Publication
(10) Pub. No.: US 2009/0118815 A1
(43) Pub. Date: May 7, 2009

(54) STENT

(75) Inventors: Ben Arcand, Minneapolis, MN (US); Tom Holman, Princeton, MN (US); James Lee Shippy, III, Maple Grove, MN (US); Liliana Atanasoska, Edina, MN (US)

Correspondence Address: FISH & RICHARDSON PC, P.O. BOX 1022, MINNEAPOLIS, MN, 55440-1022, US

(73) Assignee: Boston Scientific Scimed, Inc., Maple Grove, MN (US)

(21) Appl. No.: 11/934,463

(22) Filed: Nov. 2, 2007

(51) Int. Cl. A61F 2/06

(52) U.S. Cl. 623/1.15; 623/1.49

(57) ABSTRACT

Endoprosthesis includes coatings of selected porosity formed of particulates of ceramics, metals, drugs and/or polymers.

### TECHNICAL FIELD

This invention relates to endoprostheses such as stents.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721, the entire contents of which are hereby incorporated by reference herein.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

In an aspect, the invention features a method of forming a medical endoprosthesis stent, including entraining particles of metal, ceramic, polymer or therapeutic agent(s), or any combination thereof, in a gas stream, impacting the entrained particles on a substrate to form a deposit, and utilizing the deposit in a medical endoprosthesis.

In an aspect, the invention features an endoprosthesis, having a porous matrix of fused metal, polymer and/or ceramic particles, where the matrix has zones of different porosity.

In another aspect, the invention features a method of forming an endoprosthesis that includes entraining particles using CGDS, impacting the entrained particles to form a deposit on a substrate, and utilizing the deposit and the substrate in a medical endoprosthesis.

Embodiments may also include one or more of the following features. The particles can be entrained by CGDS. The particles can be fused to the substrate and/or each other. The particles can be fused to the substrate by melting. The particles can maintain physical integrity to form a matrix of fused particles. The porous deposit can have void regions. The porosity of the porous deposit can be controlled by controlling the velocity of the particles and/or the size of the particles and/or the temperature of the particles on the substrate. The deposit can have variable porosities as a function of thickness of the deposit. The deposit can have a region of larger voids at greater depth and a region of smaller voids at lesser depth. The deposit can include multiple regions of greater and smaller voids. The particles of different materials can be entrained simultaneously. At least one of the materials can be a therapeutic agent. A therapeutic agent containing a polymer layer can be formed over the deposit. A porous polymer layer can be formed with therapeutic agent within the pores of the porous polymer.

Embodiments may also include one or more of the following features. The zones of different porosity can be arranged as a function of depth. The endoprosthesis can include a region of greater porosity at greater depth and a region of lesser porosity at lesser depth. The matrix can include a therapeutic agent. The endoprosthesis can include a layer of polymer over the deposit. The matrix can be substantially free of polymer. The outermost deposit can be an inorganic material.

Embodiments may include one or more of the following advantages. A stent can be provided having a desired porosity and/or surface texture by a controlled deposition of particles. The process of deposition can be conducted at low temperatures, e.g. room temperature which reduces the heating of an underlying stent substrate and the deposited materials themselves. The particles can be fused to form a unitary porous body. The porosity can be tuned, e.g. as a function of depth and location to modulate the release of a therapeutic agent from the porous structure. The surface texture can be varied as a function of location to, e.g. provide a texture that encourages endothelial growth on selective surface locations (i.e. luminal, abluminal, and/or cut face) and/or enhances retention of a therapeutic agent (and its polymer carrier) coating on selective surface locations (i.e. luminal, abluminal, and/or cut face). In particular, adhesion can be enhanced for coatings that do not continuously surround a stent strut such as, e.g. a coating on only the abluminal or luminal surface. Therapeutic agents can be provided in the porous structure whereby the agent loading and its gradient as well as the porosity and its concentration gradient can be controlled to provide a desired release rate. The therapeutic agent can be coloaded with the particulates during deposition or post loaded into the formed porosity. In embodiments, a stent having desired surface texture and/or porosity can be provided that contains a therapeutic agent without a polymer carrier.

Further aspects, features, and advantages follow.

### DESCRIPTION OF DRAWINGS

FIGS. 1A-1C are cross-sectional schematics illustrating delivery of a stent into a body lumen.

FIG. 2 is a schematic perspective view of a stent.

FIG. 3A is a cross-sectional view of a stent, and FIG. 3B is a greatly enlarged view of region B in FIG. 3A.

FIG. 4 is a schematic of a system for forming a deposit on a stent surface.

FIGS. 5A-5D are cross-sectional views illustrating a method for forming a deposit on a stent surface.

FIG. 6 is an enlarged cross-sectional view of a region of a stent surface.

FIG. 7 is an enlarged cross-sectional view of a region of a stent surface.

FIG. 8 is an enlarged cross-sectional view of a region of a stent surface.

### DETAILED DESCRIPTION

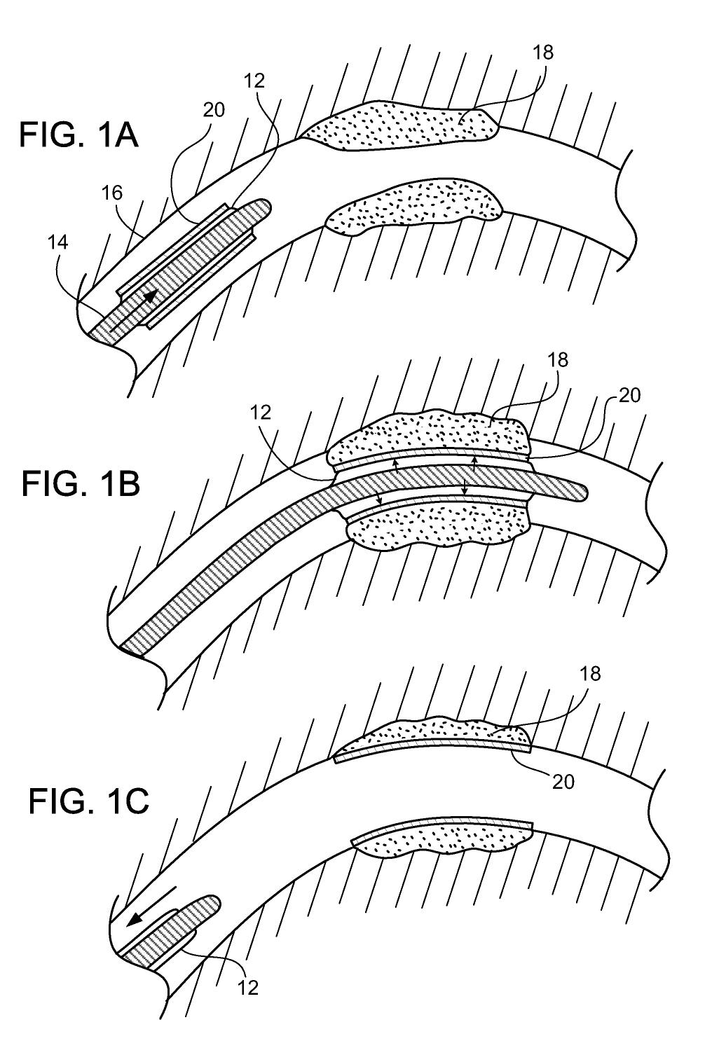

Referring to FIGS. 1A-1C, a stent 20 is placed over a balloon 12 carried near a distal end of a catheter 14, and is directed through the lumen 16 (FIG. 1A) until the balloon and stent reaches the region of an occlusion 18. The stent 20 is then radially expanded and opposed against the vessel wall by inflating the balloon 12. The occlusion 18 is opened by the opposition of the stent against the surrounding tissue/plaque. The vessel wall surrounding the occluded area undergoes a radial expansion (FIG. 1B). The pressure is then released from the balloon and the catheter is withdrawn from the vessel (FIG. 1C).

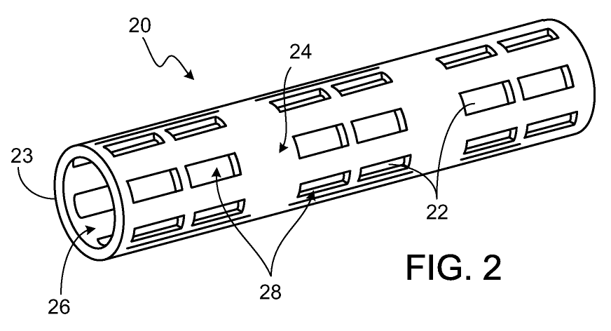

Referring to FIG. 2, the stent 20 includes a plurality of open cells surrounded by interconnecting struts 23. Stent 20 includes several surface regions, including an outer, or abluminal, surface 24, an inner, or adluminal, surface 26, and a plurality of cutface surfaces 28. The stent and its deployment mechanism can be balloon expandable, as illustrated above, a self-expanding stent, ratcheting, or by any other mechanical or fluidic means. Examples of stents are described in Heath '721, supra.

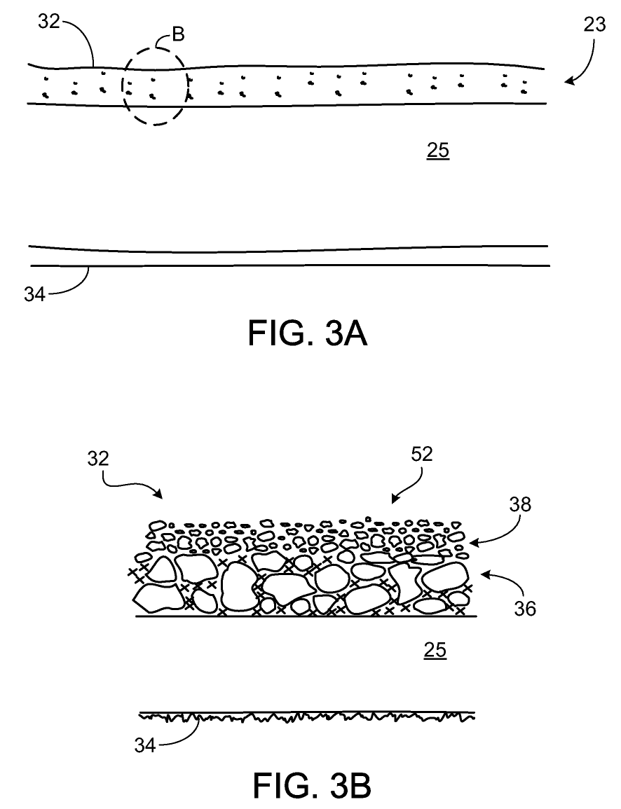

Referring to FIG. 3A, a cross-sectional view, a stent wall 23 includes a stent strut segment 25 formed, e.g. of a metal, and includes a first ceramic coating 32 on one side, e.g. the abluminal side, and a second ceramic coating 34 on the other side, e.g. the luminal side.

Referring as well to FIG. 3B, the abluminal coating 32 is formed of fused particles formed of ceramic. The coating includes an inner zone 36 having relatively large regions which contain a therapeutic agent (x's). The coating also includes an outer zone 38 having relatively small particles having relatively small void regions. In this example, the outer zone 38 serves as a diffusion membrane that controls the release of the therapeutic agent(s) from the inner zone 36 when the stent is implanted.

In embodiments, the particles in the zones can have diameters as small as submicron and as large as 50 microns. In embodiments, the size of the particles in the zones can vary. For example, the particles in the inner zone may be larger than the particles in the outer zone. In embodiments, the particles in the inner zone predominantly in the range of about 1 to 5 microns and the particles in the outer zone may be predominantly about 10 to 100 nm. The coating can have an overall thickness of about 0.5 to 10 microns. The inner zone can have a thickness ranging from 0.5 to 10 microns. The outer zone may be thinner than the inner zone. The outer zone can have a thickness ranging from 100 nm to 1 micron. The particles substantially maintain structural integrity and thus form a relatively rough granular structure and morphology on the abluminal surface of the stent.

The luminal coating 34 is also formed of fused particles. In embodiments, the luminal coating includes a single zone which has a different porosity and surface texture than the abluminal coating. For example, the particles are highly deformed and adhered to one another over a larger surface area, altering the particle geometry and displacing void spaces. The inner coating has a surface area that has a texture which facilitates endothelialization of the stent, as will be discussed further below. In other embodiments, the luminal coating has a variable porosity that is the same or different than the porosity of the abluminal coating.

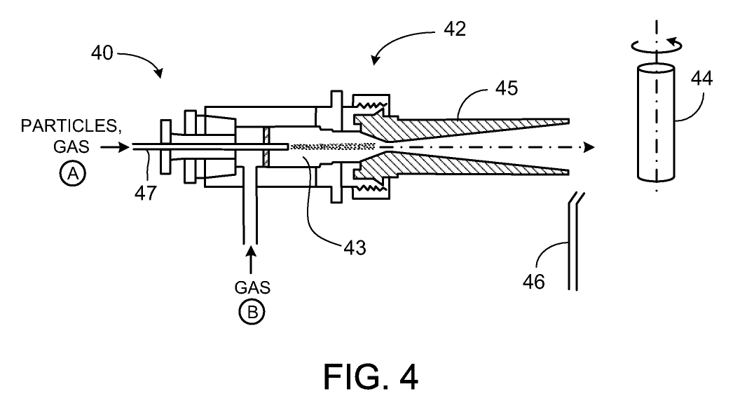

Referring to FIG. 4, the coatings are formed utilizing low temperature cold gas dynamic spraying (CGDS). In a CGDS apparatus 40, a nozzle system 42 is provided in which particles are entrained in a high velocity gas stream. Particles are carried by a gas, A, through a conduit 47 to a chamber 43 where they are entrained in a high pressure gas flow, B, immediately before or after the throat of a nozzle. The particles, traveling at high velocities, are directed to a substrate 44, such as a stent or a precursor element to a stent such as a metal tube, metal sheet or any other substrate that will subsequently be utilized in forming a stent. The particles can be accelerated to high velocities, e.g. 10 to 1300 m/sec. or more, e.g. velocities in the supersonic range, such as up to about 5000 m/sec., and at a relatively low temperature, e.g. below the melting point of the particulate material, so that particles, e.g. including the therapeutic agent and/or its polymer carrier, do not degrade, or substantially change the substrate, and the bulk of the particle does not undergo phase change. Particles can be injected pre or post nozzle depending on such aspects as the application and the position temperature of the gas. On impact, the particles adhere with the substrate and each other. The mechanism of adherence may include, for example, mechanical adherence due to plastic deformation caused by the impact, partial melting and microwelding of the particles caused by transition of kinetic to mechanical energy, and/or the physical chemical interaction of particles between other fused and bound particles. The CGDS apparatus can be used to deposit metals, ceramics, polymers, therapeutic agents, or combinations and these materials can be bonded without additional heat. Combinations of materials can be premixed before entraining in the gas stream. Alternatively or in addition, a secondary feed 46 can be used to add material to the stream. Further discussion of the CGDS technique is provided in VanSteenkiste et al., Surface and Coatings Technology 111 (1999), 62-71; Zhao et al., Surface and Coating Technology 200 (2006), 4746-4754; Easley et al., J. Mater. Res., 18(4) (2003) 855; U.S. Pat. No. 6,139,913; EI 5302414; U.S. 2006/0275554A1.

The characteristics of the deposit such as its porosity, and texture are selected to enhance the therapeutic effect of the stent by controlling the spraying characteristics, such as the velocity of the particles, the size of the particles, the temperature of the particles and the substrate, and the composition of the particles. Higher velocities, softer particles, higher temperatures cause generally greater plastic deformation on impact, resulting in denser packing of the particles which leads to void space displacement. The parameters can be controlled independently and in combination to tailor porosity and texture. The mixture of particles and/or the spray conditions can be varied as the film is being formed. For example, the velocity of ceramic and/or metal particles may be higher initially to enhance adhesion to the stent body with a thin low porosity layer and subsequently, a lower velocity is used to increase porosity while codepositing a drug. The mixture of particles can be selected to vary the drug type or concentration as a function of coating thickness. In embodiments, a polymer is codeposited with more brittle material, e.g. ceramic, to form a mixture of ceramic in a polymer. A drug can be incorporated in a polymer and/or within pores of a porous polymer deposit.

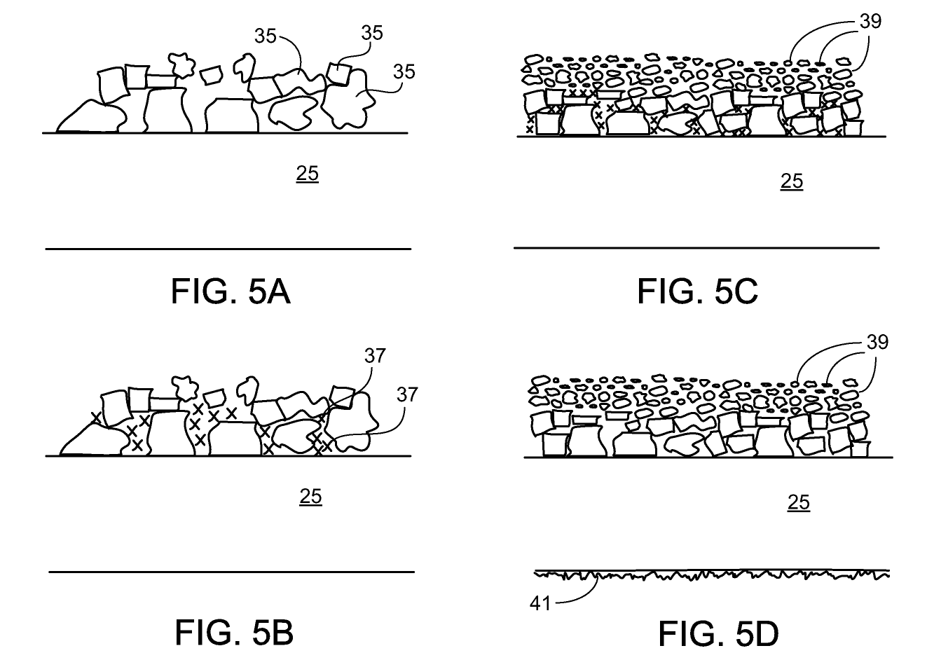

Referring to FIG. 5A, to form, for example, a stent with a combination of coatings described in FIGS. 3A and 3B, particles 35 are first deposited abluminally to form a high porosity deposit. For example, particles are deposited at sufficient velocity to adhere to particles to the substrate and each other, resulting in both adhesion and cohesion while maintaining relatively large void volume. The concentration of particles and thickness of the deposit is also selected to form a zone having substantial void space. In embodiments, relatively large particles are used. The zone can include void regions having a cross section of about 1 μm or more, e.g. 5 to 15 μm. The particles can be deposited only on the abluminal side of the stent by, e.g. masking the adluminal side, e.g. by placing the stent on a mandrel.

Referring to FIG. 5B, a therapeutic agent 37 (x's) is applied to the porous deposit to load the void cavities and form a therapeutic agent reservoir. The drug can be applied by kinetic spraying simultaneously with the particles used to form the first zone or subsequently. The therapeutic agent can also be applied by non-kinetic spraying techniques such as dipping or spraying a solution of therapeutic agent, followed by solvent evaporation.

Referring to FIG. 5C, particles 39 are deposited abluminally to form a lower porosity deposit. The lower porosity deposit is formed over the high porosity deposit by utilizing higher velocities, and/or softer particle materials to more densely pack the particles to form generally fewer and/or smaller voids. In embodiments, the particles 39 may also be relatively small compared to the particles 37. The deposited particles 39 in the lower porosity deposit tend to partially close the larger voids of the inner zone. By selecting the porosity characteristics of the outer zone, a desirable release profile of the drug is enhanced. For example, a more porous, thinner outer zone permits more rapid release while a less porous, thicker outer zone reduces the rate of therapeutic agent release.

Referring to FIG. 5D, particles 41 are then deposited luminally to form a selected roughness. For example, the luminal deposit is less porous than the outer zone of the abluminal coating. The luminal coating can be formed by very high velocity deposition to form a relatively continuous coating but with a desirable roughness to enhance endothelialization.

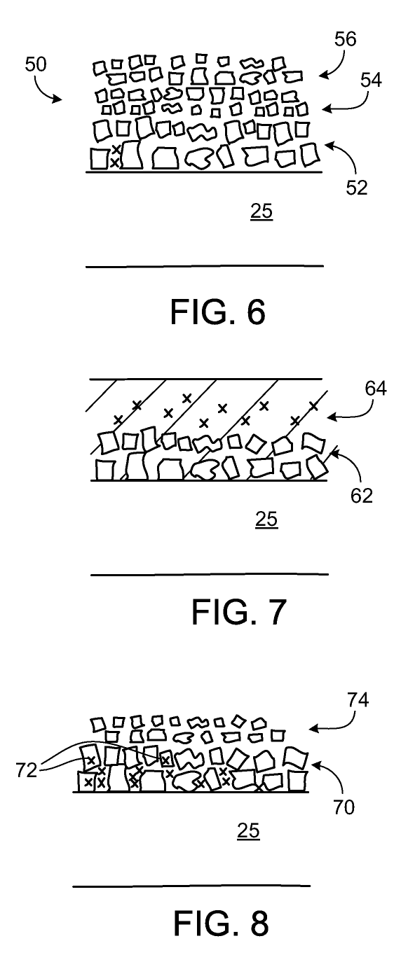

Referring to FIG. 6, in embodiments, a layer 50 has porosity varied in a plurality of zones to permit a multiphase release of drugs. For example, an inner high porosity zone 52, an inner low porosity zone 54, and an outer high porosity zone 56. The high porosity zones include therapeutic agents. Therapeutic agents (x's) in the outer high porosity zone 56 is released first at a high rate. After the therapeutic agent in the initial high porosity zone 52 is released, the drug in the inner high porosity zone is released at a lower rate, modulated by the low porosity zone 54. In other embodiments, an outer low porosity zone can be provided over the outer high porosity zone to modulate the rate of therapeutic agent release, at the same or different rates than the inner low porosity zone. The therapeutic agents in the inner and outer high porosity zones can be the same or different. Therapeutic agents can also be provided in the low porosity zone. In embodiments, the stent can be substantially free of polymer or polymer layers on its surface allowing direct delivery of therapeutic agents from porous zones formed of non-polymer materials such as metals and/or ceramics.

Referring to FIGS. 7 and 8, in embodiments, the deposited materials can be used in combination with polymers. Referring particularly to FIG. 7, a stent body includes a deposit of particles 62, such as metal or ceramics that form a high roughness surface. A polymer layer 64, e.g. including a therapeutic agent, is applied over the high roughness deposit. The polymer layer can be applied by dynamic spraying or by solvent spraying or dipping. The high roughness of the deposit enhances the adhesion of the polymer to the stent.

Referring to FIG. 8, in embodiments, a porous deposit 72 is formed on a stent surface and polymer particles, e.g. including therapeutic agents, are applied to the voids of the deposit. The polymer particles can be applied by dynamic spraying or by spraying in a solvent or dipping. A low porosity deposit 76 or a polymer membrane can be applied to modulate therapeutic agents released by the polymer. In embodiments, the membrane 76 is formed of an inorganic material, e.g. a ceramic or a metal such that an inorganic layer faces the blood stream.

The roughness of the surface is characterized by the average roughness, Sa, the root mean square roughness, Sq, and/or the developed interfacial area ratio, Sdr. The Sa and Sq parameters represent an overall measure of the texture of the surface. Sa and Sq are relatively insensitive in differentiating peaks, valleys and the spacing of the various texture features. Surfaces with different visual morphologies can have similar Sa and Sq values. For a surface type, the Sa and Sq parameters indicate significant deviations in the texture characteristics. Sdr is expressed as the percentage of additional surface area contributed by the texture as compared to an ideal plane the size of the measurement region. Sdr further differentiates surfaces of similar amplitudes and average roughness. Typically Sdr will increase with the spatial intricacy of the texture whether or not Sa changes.

In embodiments, the ceramic has is relatively rough, the Sdr is about 30 or more, e.g. about 40 to 60. In addition or in the alternative, the morphology has an Sq of about 15 or more, e.g. about 20 to 30. In embodiments, the Sdr is about 100 or more and the Sq is about 15 or more. In other embodiments, the ceramic is relatively less rough, the Sq is about 15 or less, e.g. about less than 8 to 14. In still other embodiments, the Sdr and Sq values between the ranges above, e.g. an Sdr of about 1 to 200 and/or an Sq of about 1 to 30. Surface roughness is further described in U.S. patent application Ser. Nos. 11/752,735 and 11/752,772, both filed May 23, 2007.

In other embodiments, the porosity or texture can be varied along the stent, e.g. in successive radial regions along the stent axis or longitudinal regions parallel to the stent axis. Different zones can be arranged for the release of different therapeutic agents and/or different release rates.

As discussed above, the particles applied can be metals, ceramics, polymers, or therapeutic agents. The porous structures can be formed by one or any combination of these materials. The materials can be body-fluid degradable or stable. Body-fluid degradable layers can be used to vary porosity or texture over time. For example, multiple layers of bioerodible material may be provided with successive layers having different porosity or texture. As an outermost layer erodes, an inner layer having a different porosity or texture is exposed.

Suitable ceramics include metal oxides and nitrides such as of iridium, zirconium, titanium, hafnium, niobium, tantalum, ruthenium, platinum, and aluminum. Suitable metals include biostable or bioerodible metals, e.g. magnesium. Suitable metals include stainless steels, platinum, gold, titanium, magnesium, iron, including alloys and metal-metal and metal-non-metal capacity composites. In embodiments, the deposit can be enhanced by radiopacity of the stent by including radiopaque metals. Radiopaque metals are discussed in Heath, supra. In embodiments, the particles are hollow spheres or fibers, e.g. formed of ceramic, e.g. biodegradable ceramic including a drug. The ceramic does not substantially conduct heat, reducing the likelihood of degradation of the drug. Hollow spheres are described in U.S. Ser. No. ______, filed ______ [Atty. Docket No. 10527-802P01.]

Suitable polymers may be biostable or biodegradable. Suitable polymers include, for example, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics such as polystyrene and copolymers thereof with other vinyl monomers such as isobutylene, isoprene and butadiene, for example, styrene-isobutylene-styrene (SIBS), styrene-isoprene-styrene (SIS) copolymers, styrene-butadiene-styrene (SBS) copolymers, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenerated polyalkylenes including polytetrafluoroethylene, natural and synthetic rubbers including polyisoprene, polybutadiene, polyisobutylene and copolymers thereof with other vinyl monomers such as styrene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyiocyanates such that the devices become instantly lubricious when exposed to body fluids. In another preferred embodiment of the invention, the polymer is a copolymer of polylactic acid and polycaprolactone. Suitable polymers are discussed in U.S. Publication No. 20060038027.

The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. Polymers for drug elution coatings are also disclosed in U.S. Published Patent Application No. 2005/019265A. A functional molecule, e.g. an organic, drug, polymer, protein, DNA, and similar material can be incorporated into groves, pits, void spaces, and other features of the ceramic.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, uretheral lumens and ureteral lumens.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, Biodur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721). The ceramics can be used with other endoprostheses or medical devices, such as catheters, guide wires, and filters.

All publications, patent applications, patents, and other references mentioned herein including the appendix, are incorporated by reference herein in their entirety.

Still other embodiments are in the following claims.

What is claimed is:

1. A method of forming a medical endoprosthesis, comprising:
entraining particles of metal, ceramic, polymer, therapeutic agent(s) or any combination thereof in a gas stream,
impacting the entrained particles on a substrate to form a deposit, and
utilizing the deposit in a medical endoprosthesis.

2. The method of claim 1, comprising entraining said particles using CGDS.

3. The method of claim 1, wherein the particles are fused to the substrate and/or each other.

4. The method of claim 3, wherein the particles are fused to the substrate by melting.

5. The method of claim 4, wherein the particles maintain physical integrity to form a matrix of fused particles.

6. The method of claim 1, comprising forming a porous deposit having void regions.

7. The method of claim 6, comprising controlling the porosity of the porous deposit by controlling the velocity of the particles and/or the size of the particles and/or the temperature of the particles on the substrate.

8. The method of claim 7, comprising forming a deposit having variable porosities as a function of thickness of the deposit.

9. The method of claim 8, wherein the deposit has a region of larger voids at greater depth and a region of smaller voids at lesser depth.

10. The method of claim 9, wherein the deposit includes multiple regions of greater and smaller voids.

11. The method of claim 1, comprising simultaneously entraining particles of different materials.

12. The method of claim 11, wherein at least one of the materials is a therapeutic agent.

13. The method of claim 1, comprising forming a therapeutic agent containing polymer layer over the deposit.

14. The method of claim 1, comprising forming a porous polymer layer with therapeutic agent within the pores of the porous polymer.

15. An endoprosthesis, comprising:
a porous matrix of fused metal, polymer and/or ceramic particles, the matrix having zones of different porosity.

16. The endoprosthesis of claim 15, wherein the zones of different porosity are arranged as a function of depth.

17. The endoprosthesis of claim 16 including a region of greater porosity at greater depth and a region of lesser porosity at lesser depth.

18. The endoprosthesis of claim 15, wherein the matrix includes a therapeutic agent.

19. The endoprosthesis of claim 15 including a layer of polymer over the deposit.

20. The endoprosthesis of claim 15, wherein the matrix is substantially free of polymer.

21. The endoprosthesis of claim 15, wherein the outermost deposit is an inorganic material.

22. A method of forming an endoprosthesis, comprising:
entraining particles using CGDS,
impacting the entrained particles to form a deposit on a substrate, and
utilizing the deposit and the substrate in a medical endoprosthesis.
